(19) United States
(12) Patent Application Publication
(10) Pub. No.: US 2024/0135168 A1
(43) Pub. Date: Apr. 25, 2024

(54) PEROVSKITE SYNTHESIZABILITY PREDICTION METHOD USING GRAPH CONVOLUTIONAL NEURAL NETWORKS AND POSITIVE UNLABELED LEARNING

(71) Applicant: KOREA ADVANCED INSTITUTE OF SCIENCE AND TECHNOLOGY, Daejeon (KR)

(72) Inventors: You Sung JUNG, Daejeon (KR); Geun Ho GU, Daejeon (KR); Ju Hwan NOH, Daejeon (KR); Ji Don JANG, Daejeon (KR)

(21) Appl. No.: 18/091,877

(22) Filed: Dec. 30, 2022

(30) Foreign Application Priority Data
Oct. 18, 2022 (KR) 10-2022-0134516

(51) Int. Cl. G06N 3/08

(52) U.S. Cl. CPC G06N 3/08

(57) ABSTRACT

Provided is a method for predicting perovskite synthesizability using a graph convolutional neural network and positive unlabeled learning, capable of predicting perovskite synthesizability by using a graph convolutional neutral network and positive unlabeled learning which is semi-supervised learning based on a labeled model using positive data and positive unlabeled data.

### CROSS REFERENCE TO RELATED APPLICATION

The present application claims priority to Korean Patent Application No. 10-2022-0134516, filed on Oct. 19, 2022, the entire contents of which is incorporated herein for all purposes by this reference.

### BACKGROUND OF THE INVENTION

### Field of the Invention

The disclosure relates to perovskite synthesizability prediction, and more particularly, a method for predicting perovskite synthesizability using a graph convolutional neural network and positive unlabeled learning, which is semi-supervised learning based on a labeled model using positive data and positive unlabeled data.

### Description of the Related Art

The discovery of new functional material is a major goal of a materials science. Advances in electronic structure computation and the development of digital crystal databases have led to the successful discovery of several new functional materials through high-throughput screening (HTS).

HTS is typically performed in hierarchical steps, starting with density functional theory (DFT) database screening of previously synthesized materials, followed by high-level DFT refinement and experimental validation, performed in hierarchical steps of increasing accuracy and cost. To ascertain the scope, databases such as Materials Project (MP) 7, OQMD and AFLOW have collected a large number of unlabeled crystals that have in silico ground-state structures but have not yet been synthesized experimentally. Some of the promising unlabeled crystals have been actually synthesized, demonstrating the feasibility of virtual screening strategies to discover new materials.

Since many, if not most, of the virtual materials screened have not been experimentally realized, evaluating their synthesizability has been an important challenge. In general, the synthesizability of virtual materials is evaluated using the energy above the convex hull.

However, as is well known, the latter thermodynamic measurement method is not sufficient to evaluate the synthesizability because the synthesis kinetics and growth conditions such as precursor selection, annealing temperature and duration, and external pressure are largely neglected. Therefore, a generalized and more reliable method for evaluating the synthesizability of candidate crystals is needed.

In addition, perovskite is attracting more and more attention for applications in photovoltaics, light emitting diodes, magnetic materials, superconductors, and lithium ion conductors, and the like. This perovskite is an important material type for geophysical and technologically important applications, but there are relatively few known perovskites. Therefore, a method for discovering efficient materials by developing a perovskite-focused model with improved accuracy is required.

### SUMMARY OF THE INVENTION

Therefore, in order to solve the above-mentioned problems of the prior art, a technical object of an embodiment of the disclosure is to provide a method for predicting perovskite synthesizability using a graph convolutional neural network and positive unlabeled learning, capable of predicting perovskite synthesizability by using a graph convolutional neural network and positive unlabeled (PU) learning which is semi-supervised learning based on a labeled model using positive data (synthesizable data) and positive unlabeled (PU) data.

The problems to be solved in the disclosure are not limited to the problems mentioned above, and other problems not mentioned may be clearly understood by those skilled in the art from the following descriptions.

In order to achieve the above object of the disclosure, an embodiment of the disclosure provides a method for predicting perovskite synthesizability using a graph convolutional neural networks and positive unlabeled learning, including a graph convolutional neural network model pre-training step of performing pre-training for perovskite synthesizability prediction by inputting stored material data into a graph convolutional neural network model that calculates a perovskite synthesizability score; a graph convolutional neural network model retraining step of performing retraining for the perovskite synthesizability prediction by inputting stored perovskite data to the graph convolutional neural network model; and a perovskite synthesizability prediction step of calculating the perovskite synthesizability score by randomly selecting unlabeled data from perovskite data set as negative data and then applying the selected unlabeled data to the retrained graph convolutional neural network model.

The graph convolutional neural network model in each step may be configured to receive an atomic feature and an edge feature of each of the material data and the perovskite data as an input value and calculate the synthesizability score of the material or the perovskite.

The graph convolutional neural network model pre-training step may be the step of pre-training the graph convolutional neural network model by positive unlabeled learning that repeatedly calculates a synthesizability score by applying the graph convolutional neural network model after randomly selecting unlabeled material data included in the material data for which synthesizability is not determined to set the selected unlabeled material data to a negative indicating synthesis impossibility.

The graph convolutional neural network model retraining step may be the step of retraining the graph convolutional neural network model by positive unlabeled learning that repeatedly calculates a synthesizability score by applying the pre-trained graph convolutional neural network model after randomly selecting unlabeled perovskite data included in the perovskite data for which synthesizability is not determined to set the selected unlabeled perovskite data to a negative indicating synthesis impossibility.

The perovskite synthesizability prediction step may be the step of predicting the perovskite synthesizability by performing positive unlabeled learning that repeatedly calculates synthesizability by randomly selecting unlabeled perovskite data from the perovskite data set as the negative data and then inputting the selected unlabeled perovskite data to the retrained graph convolutional neural network model, and averaging the synthesizability score for each perovskite data calculated in each data set by the positive unlabeled learning.

The perovskite synthesizability prediction step may predict as being synthesizable when a predicted perovskite synthesizability score is 0.5 or more.

Another embodiment of the disclosure provides a recording medium in which the method for predicting perovskite synthesizability is recorded as a code that is read and executed by a computer.

The above-described embodiment of the disclosure, in predicting the perovskite synthesizability, showed a high positive data accuracy of out-of-sample of 95.7% compared to the accuracy of the non-domain specific original model of about 74.0%.

In addition, an embodiment of the disclosure can have an effect of predicting the synthesizability of all types of perovskites in the data set, including anti-perovskites in which anion and cation occupancy are reversed, compared to the conventional ionic perovskite-focused model.

In addition, an embodiment of the disclosure provides the effect of predicting Li-rich anti-perovskites and metal halides as promising candidates for the discovery of solid electrolytes and photoactive materials, respectively.

It is to be understood that the effect of the disclosure is not limited to the above-described effects, and includes any effects deducible from the features of the disclosure described in the detailed description or the claims of the disclosure.

### BRIEF DESCRIPTION OF THE DRAWINGS

FIG. 1 is a flow chart showing the processing of a method for predicting perovskite synthesizability using a graph convolutional neural network and positive unlabeled learning of an embodiment of the disclosure.

FIG. 2 is a diagram showing the learning and synthesizability prediction processing concept of a perovskite synthesis method using a graph convolutional neural network and positive labeled learning according to an embodiment of the disclosure.

FIG. 3 is a diagram showing (a) accuracy and (b) calculated synthesizable score distribution of a perovskite synthesis method using a graph convolutional neural network and positive unlabeled learning of an embodiment of the disclosure.

FIG. 4 is a diagram for verifying a perovskite synthesis method using a graph convolutional neural network and positive unlabeled learning of an embodiment of the disclosure.

FIG. 5 is a diagram showing the synthesizability of ABO₃ perovskite compound (lower left triangle) predicted by a perovskite synthesis method using a graph convolutional neural network and positive unlabeled learning of an embodiment of the disclosure and the synthesizability of ABO₃ perovskite compound predicted (upper right triangle) by Goldschmidt rule-based screening.

FIG. 6 is a graph showing a prediction result (a) according to an embodiment of the disclosure and prediction results (b, c, d) of other methods for reported unlabeled perovskite.

### DETAILED DESCRIPTION OF THE INVENTION

Hereinafter, the disclosure will be described in detail with reference to the accompanying drawings. However, it should be understood that the disclosure can be implemented in various forms, and that it is not intended to limit the disclosure to the embodiments described herein. Also, in the drawings, descriptions of parts unrelated to the detailed description are omitted to clearly describe the disclosure. Throughout the specification, like numbers refer to like elements.

Throughout this specification, when a part is mentioned as being “connected (accessed, contacted, coupled)” to another part, this means that the part may not only be “directly connected” to the other part but may also be “indirectly connected” to the other part through another member interposed therebetween, in addition, when a part is mentioned as “including” a specific component, this does not preclude the possibility of the presence of other component(s) in the part Which means that the part may further include the other component(s), unless otherwise stated.

The terminology used herein is for the purpose of describing various embodiments only and is not intended to be limiting. As used herein, the singular forms are intended to include the plural forms as well, unless the context clearly indicates otherwise. It will be further understood that the terms “comprises” or “has,” when used in this specification, specify the presence of a stated feature, number, step, operation, component, element, or a combination thereof, but do not preclude the presence or addition of one or more other features, numbers, steps, operations, components, elements, or combinations thereof.

Hereinafter, embodiments of the disclosure will be described in detail with reference to the accompanying drawings.

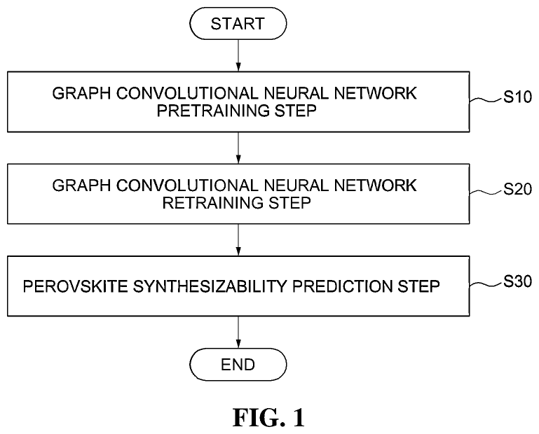

FIG. 1 is a flow chart showing the processing of a method for predicting perovskite synthesizability using a graph convolutional neural network and positive unlabeled learning (hereinafter “perovskite synthesizability prediction method”) of an embodiment of the disclosure.

As shown in FIG. 1, a perovskite synthesizability prediction method may be configured to include a graph convolutional neural network pretraining step (S10), a graph convolutional neural network retraining step (S20), and a perovskite synthesizability prediction step (S30).

The graph convolutional neural network pretraining step (S10) may be the step of performing pretraining for predicting perovskite synthesizability by inputting stored material data to a graph convolutional neural network model that calculates a perovskite synthesizability score.

The graph convolutional neural network model pretraining step (S10) may be the step of pretraining the graph convolutional neural network model by positive unlabeled (PU) learning that repeatedly calculates a synthesizability score by applying the graph convolutional neural network model after randomly selecting unlabeled material data included in the material data for which synthesizability is not determined to set the selected unlabeled material data to a negative indicating synthesis impossibility.

The graph convolutional neural network retraining step (S20) may be the step of performing retraining for predicting perovskite synthesizability by inputting stored perovskite data into the graph convolutional neural network model.

The graph convolutional neural network model retraining model (S20) may be the step of retraining the graph convolutional neural network model by positive unlabeled learning that repeatedly calculates a synthesizability score by applying the pretrained graph convolutional neural network model after randomly selecting unlabeled perovskite data included in the perovskite data for which synthesizability is not determined to set the selected unlabeled perovskite data to a negative indicating synthesis impossibility.

In the above perovskite synthesizability prediction step (S30) may be the step of calculating the perovskite synthesizability score by randomly selecting unlabeled data from the perovskite data set as negative data, and then applying the selected unlabeled data to the retrained graph convolutional neural network model.

The perovskite synthesizability prediction step (S30) may be the step of predicting the perovskite synthesizability by performing positive unlabeled learning that repeatedly calculates synthesizability by randomly selecting unlabeled perovskite data from the perovskite data set as the negative data and then inputting the selected unlabeled perovskite data to the retrained graph convolutional neural network model, and averaging the synthesizability score for each perovskite data calculated in each data set by the positive unlabeled learning.

In the perovskite synthesizability prediction step (S30), if the predicted perovskite synthesizability score is 0.5 or more, synthesizability may be predicted.

The graph convolutional neural network model of each step (S10, S20, and S30) may be configured to receive the atomic feature and edge feature of each of the material data and perovskite data as input values, and calculate the synthesizability score of the material or perovskite.

### Experimental Example

The graph convolutional neural network model was pretrained with the material data stored in the Materials Project (MP) database, and the graph convolutional neural network model was retrained with smaller perovskite data sets stored in the MP, OQMD, and AFLOW databases.

943 perovskite crystals synthesized in the prior art (positive) and 11,964 unlabeled (virtual) perovskite data with undetermined synthesizability collected from MP, OQMD and AFLOW databases were used for learning.

The perovskite synthesizability prediction method using a graph convolutional neural network model and positive unlabeled learning of the embodiment of the disclosure has a high positive data accuracy of out-of-sample of 95.7% compared to the accuracy of the non-domain specific original model of about 74.0%.

The perovskite synthesizability prediction method of the disclosure predicted that 962 materials among 11,964 unlabeled perovskites could be synthesized. Among them, 179 unlabeled crystals (virtual crystals) were actually synthesized in the literature. Compared to a conventional ionic perovskite-focused model, the perovskite synthesizability prediction method of the disclosure may predict the perovskite synthesizability of all types of data sets, including anti-perovskite in which anion and cation sites are reversed. The perovskite synthesizability prediction method according to the disclosure could suggest Li-rich anti-perovskite and metal halide as new promising candidate materials for the discovery of solid electrolytes and photoactive materials, respectively.

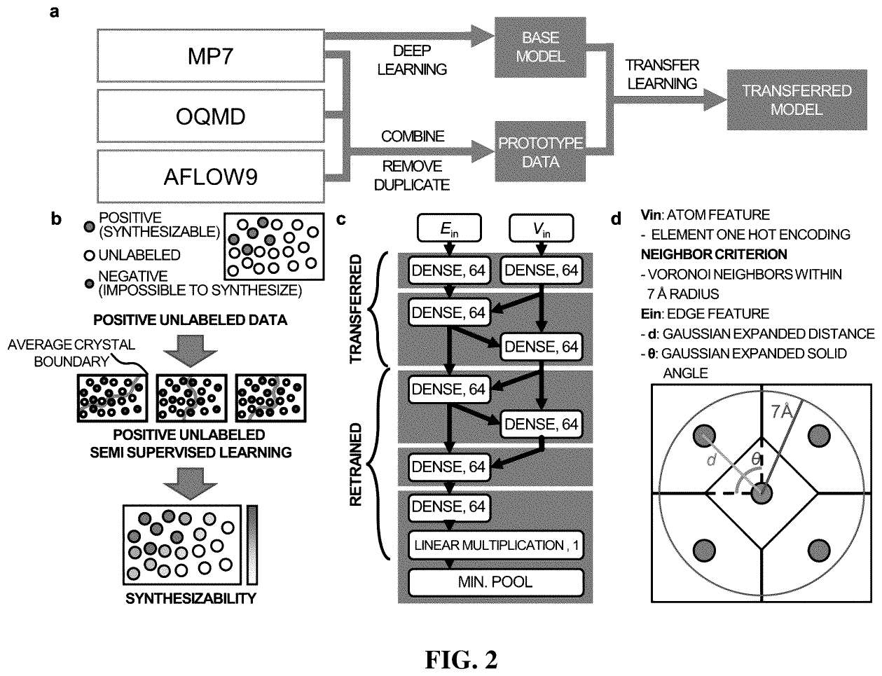

FIG. 2 is a diagram showing the learning and synthesizability prediction processing concept of a perovskite synthesis method using a graph convolutional neural network and positive labeled learning according to an embodiment of the disclosure.

In FIG. 2, a shows a transfer learning flow, b shows an overview of the positive unlabeled (PU) learning procedure, c shows a graph convolutional neural network architecture, d shows a method for mathematical transformation of the atomic feature and edge feature input to the graph convolutional neural network.

In c of FIG. 2, the “dense” box represents a softplus activation layer after linear multiplication. The “Linear” box represents linear multiplication. The number next to “dense” or “linear” indicates an output feature dimension. Min Pool represents minimum pooling followed by sigmoid activation.

As shown in a of FIG. 2, the perovskite synthesizability prediction method of an embodiment of the disclosure is trained with the Materials Project (MP) database, and the model is trained again (retrained) with perovskite-dedicated data extracted from three databases.

Specifically, the inorganic crystal data of the MP database retrieved in October 2020 consists of 46,546 crystals with inorganic crystal structure database (ICSD) ids and 79,789 crystals without ICSD ids. 46,546 crystals with ICSD id and experimental tag can be synthesized. The remaining 79,789 crystals without an ICSD id were “unlabeled (virtual)” and considered not to be synthesizable. This MP data is used to pre-train the model.

Then, the perovskite crystals were retrieved in the MP, OQMD and AFLOW databases in October 2020 (a in FIG. 2). The duplicate crystals were identified and removed using the structure matcher function of pymatgen (Python Materials Genomics) and perovskite prototype structures of the AFLOW database, thereby generating 943 synthesized crystals and 11,964 unlabeled perovskite crystals. The generated perovskite data is used to train a transfer model.

Both the pre-training and the transfer learning are performed using inductive PU learning. In order to test the perovskite synthesizability prediction method using the graph convolutional neural network and the positive unlabeled learning model of the disclosure, 10% of synthetic crystals are set to be randomly sampled from both the MP data used for pre-training and the perovskite data used for transfer learning. Therefore, test data is not observed in the pre-training step. The PU learning procedure is performed with the remaining data set. Here, 10% of synthesized crystals are randomly sampled and the same number of unlabeled (virtual) crystals are randomly sampled for model validation. The remaining synthesized crystals are used for training, and the same number of unlabeled crystals are randomly sampled and processed as negative (impossible to synthesize) data for learning.

This process is repeated 100 times to create an ensemble of 100 models.

It is the characteristic of the disclosure that for each model the training and validation set for unlabeled crystal is changed while the training and validation set for synthetic crystal remains fixed.

The synthesizability score, called a crystal likeness (CL) score, is calculated by averaging the predictions of 100 models. Changing the virtual data set forms an average crystal boundary, as conceptually shown in b of FIG. 2. The average crystal boundary may be applied as a criterion for labeling synthesizable positive perovskite data and unlabeled and negative perovskite data.

To implement the perovskite synthesis method, a graph convolutional neural network (GCNN) model was constructed as shown in c of FIG. 2.

The perovskite synthesizability prediction method using the graph convolutional neural network and the positive unlabeled learning model of the disclosure calculates the CL score between 0 and 1. Here, the crystal with a high CL score indicates high synthesizability. For practical screening, the crystal candidates are tested in descending order of their CL scores, giving them the best chance of success. Specifically, by setting the CL score to 0.5, a matrix such as a true positive rate (TPR; true positives/(true positives+false negatives)) is calculated and the crystal is considered as a synthesizable candidate.

To perform transfer learning, the graph convolutional neural network model was first pre-trained by material data from the MP database. Then, the model weights of an encoding layer and a first graphic convolution layer were fixed, and the remaining models were retrained by pre-synthesized perovskite data.

<Accuracy and Verification of the Perovskite Synthesizability Method of the Disclosure Using Literature Experiments>

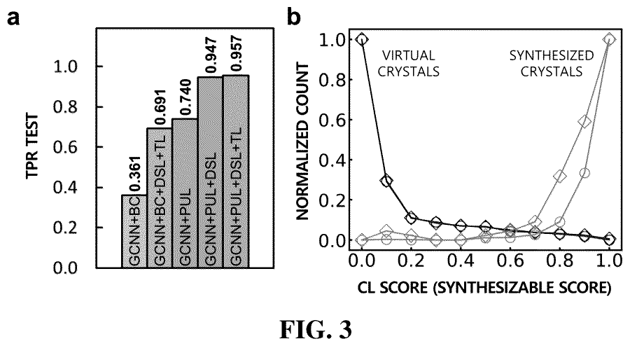

FIG. 3 is a diagram showing (a) accuracy and (b) calculated synthesizable score distribution of a perovskite synthesis method using a graph convolutional neural network and positive unlabeled learning of an embodiment of the disclosure.

In FIG. 3, a is a graph showing the true positive rates by a perovskite synthesizability prediction model of an embodiment of the disclosure and a conventional perovskite synthesizability prediction model.

GCNN represents the graph convolutional neural network model of an embodiment of the disclosure, BC represents a binary classification, PUL represents a positive unlabeled learning, DSL represents a domain specific learning, and TL represents a transfer learning. Since positive data (synthesizable crystals) are known but negative data (non-synthesizable crystals) are unknown, the true positive rate is evaluated as a performance measurement. Since unlabeled data (virtual crystal) is available in the database, positive unlabeled learning is implemented to evaluate synthesizability.

In FIG. 3, b shows a calculated synthesizability score distribution, and the diamond and circle marks represent the out-of-sample test data and all data, respectively. Counts are normalized to the highest peak value.

Each perovskite synthesis method was evaluated using the positive test perovskite data set as shown in a of FIG. 3.

Emphasis was placed on TPR because negative data (impossible to synthesize) was not available. Compared to the MP-trained general synthesizability prediction model, domain specific transfer positive unlabeled (PU) learning has a significantly higher TPR for perovskite, ranging from 0.740 (GCNN+PUL in a of FIG. 3) to 0.957 (GCNN+PUL+DSL+TL in a of FIG. 3).

For comparison, as a result of testing the crystal graph convolutional neural network (CGCNN) model in a previous study, it was found that the TPR was 0.595 and 0.957 for general model and graph neural network domain-specific transfer PU learning, respectively.

To evaluate a perovskite chemical space, the CL score distribution was plotted for unlabeled and synthetic crystals (b in FIG. 3). The score for unlabeled crystals was biased toward the CL score of 0, and only 962 out of 11,964 unlabeled perovskites (1121 considering structural distortion) were predicted to be synthesizable. It was found that domain-specific transfer learning can improve the accuracy of the oxide-centered chemical space (from 0.837 to 0.930).

In FIG. 3, b shows the CL score distribution for all data and out-of-sample test data, which also shows that unlabeled crystals are generally predicted to be non-synthesizable.

For verification of the perovskite synthesizability prediction method of the disclosure, it was tested the binary classification model trained by GCNN using a negative data set in which all unlabeled data are labeled as negative numbers, and the positive data was oversampled to balance the numbers of positive and negative data. Here, it was found that the TPR decreased to 0.361 (GCNN+BC in a of FIG. 3) and 0.691 (GCNN+BC+DSL+TL in a of FIG. 3) for the MP-trained general model and the transfer learning model, respectively. This could be because the positive data of the unlabeled data was incorrectly designated as negative numbers, so it was confirmed that the data division method was important in PU learning. Also, the PU learning model was trained without pre-training (i.e., without transfer learning) with MP data and a slight decrease in the TPR to 0.947 (GCNN+PUL+DSL in a of FIG. 3) was found. Therefore, the model success is mainly due to the domain-specific data set, and the TPR is slightly based on the transfer learning scheme.

Accordingly, the following will be described using the results obtained by the perovskite synthesizability method of an embodiment of the disclosure to which GCNN+PUL+DSL+TL, which is the best model, is applied.

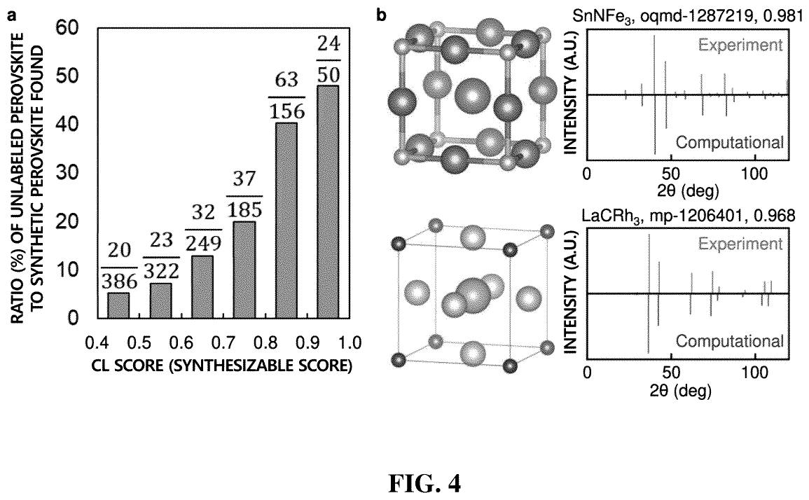

FIG. 4 is a diagram for verifying a perovskite synthesis method using a graph convolutional neural network and positive unlabeled learning of an embodiment of the disclosure.

<Validation>

In FIG. 4, a is a graph showing the ratio of unlabeled perovskite found to be synthesized in previously published literature.

The ratio of unlabeled perovskite represents the number of crystals found relative to the number of virtual crystals found within a corresponding scope.

In FIG. 4, b is a diagram showing the structure of an unlabeled crystal and XRD comparison between experiment and unlabeled crystals for the top two perovskites reported in previously published literature.

In a of FIG. 4, the plot of the percentage of unlabeled crystals found by CL score shows an interesting trend, with the proportion of previous syntheses increasing with the predicted CL score.

In FIG. 4, b shows two previously synthesized unlabeled perovskites with the highest synthesis scores and respective XRD patterns. Also, the literature was retrieved for the 1000 unlabeled crystals with the lowest CL scores, but any previous reports for their synthesis could not be found. Also, the literature was retrieved for crystals with the CL score between 0.4 and 0.5 to evaluate the model's performance on crystals with uncertain synthesizability. Among the 386 unlabeled crystals for this crystal, only 20 previously synthesized crystals were found. This represents the CL score value of the domain where it is difficult to determine synthesizability.

<Comparison with Tolerance-Based Model>

The TPR of out-of-sample of the perovskite synthesizability prediction method of the disclosure is compared with two empirical perovskite discovery strategies: Goldschmidt rule-based and SISSO-based screening.

The Goldschmidt tolerance factor-based screening was used by evaluating the ionic radius of Shannon's table (Shannon, R. Revised effective ionic radii and systematic studies of interatomic distances in halides and chalcogenides. Acta Crystallogr. Sect. A 32, 751-767 (1976)). This screening focused on standard ionic perovskites in which elements at the C site in the ABC₃ formula are restricted to seven anions.

Since the perovskite data applied to the disclosure included non-classical ionic perovskites, only 388 of the 943 synthesized perovskites were found to be within the screening scope.

For 388 perovskites, the TPR of 0.863 was obtained using Davies et al.'s method (Computational screening of all stoichiometric inorganic materials. Chem 1, 617-627 (2016)).

Bartel et al. developed and used a SISSSO-determined tolerance factor using oxidation state and ionic radius (New tolerance factor to predict the stability of perovskite oxides and halides. Sci. Adv. 5, eaav0693 (2019)).

Although only 310 crystals of the 943 perovskites were within the scope of elemental selection, the procedure was reproduced and the TPR of 0.806 was calculated.

As a result of comparison, the TPR (0.957) of out-of-sample of the perovskite synthesizability prediction method using the graph convolutional neural network and the positive unlabeled learning model of the disclosure was much higher than that (0.806 to 0.863) of a conventional method for the experimentally synthesized perovskite considered.

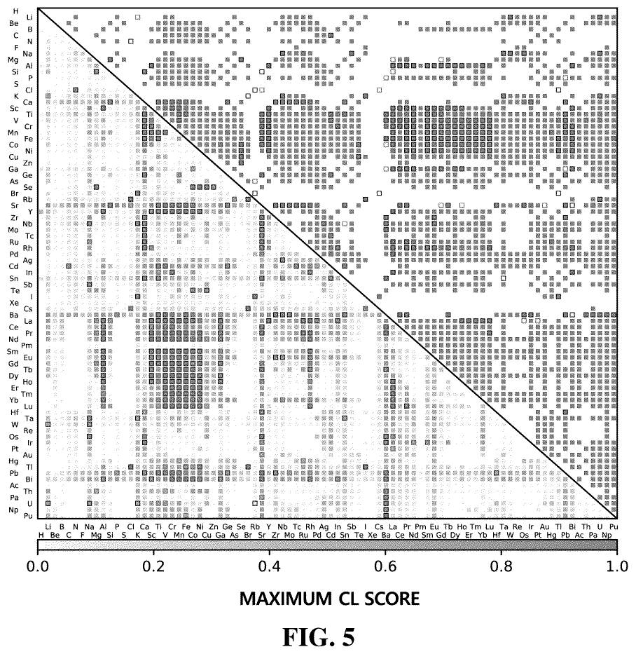

FIG. 5 is a diagram showing the synthesizability of ABO₃ perovskite compound (lower left triangle) predicted by a perovskite synthesis method using a graph convolutional neural network and positive unlabeled learning of an embodiment of the disclosure and the synthesizability of ABO₃ perovskite compound (upper right triangle) predicted by Goldschmidt rule-based screening.

The green color in the lower left triangle represents the maximum CL score for a perovskite structure in the database with a given composition. The green color in the upper right triangle represents combinations passing through the screening. A blue box represents that the combination was previously synthesized. The red box represents the unlabeled crystal found to be previously synthesized.

In addition, since the perovskite synthesizability prediction method using the graph convolutional neural network and the positive unlabeled learning model of the disclosure predicts the probability, the best candidate may be assigned priority.

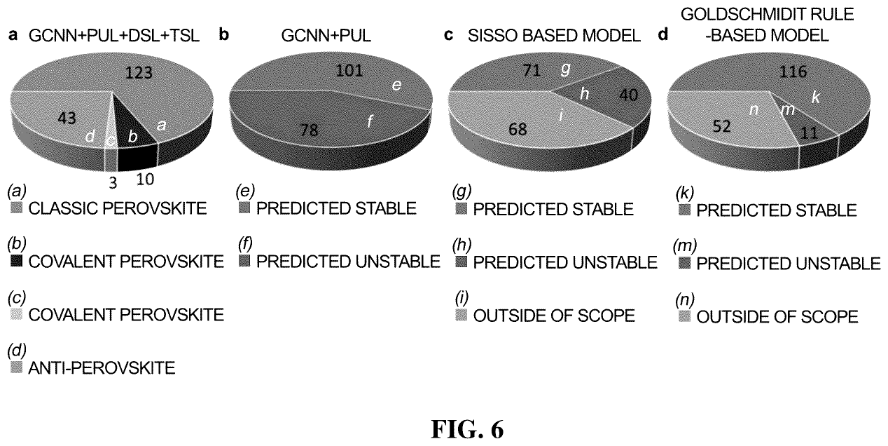

FIG. 6 is a graph showing a prediction result (a) according to an embodiment of the disclosure and prediction results (b, c, d) of other methods for reported unlabeled perovskite.

In FIG. 6, a shows a perovskite type distribution for 179 unlabeled perovskites whose synthesizability is predicted according to the perovskite synthesizability prediction method according to an embodiment of the disclosure.

In FIG. 6, b shows the perovskite synthesizability distribution predicted by a non-domain specific MP trained general model (GCNN+PUL in a of FIG. 2).

In FIG. 6, c and d show the perovskite synthesizability distributions predicted with stability of 179 compounds using SISSSO-based model and Goldschmidt rule-based screening.

ABC₃ perovskites were classified according to the following criteria.

Classical perovskites contain cations at the A and B sites and anions at the C site (e.g., SrTiO₃).

Anti-perovskite contains anion at the B site and cation at the A and C sites (e.g., SnNFe₃)),

Covalent perovskites contain two or more anions (e.g., CsIO₃, ClOLi₃) and hydrides contain hydrogen at the C site (e.g., CaCsH₃).

The perovskite synthesizability prediction method of the disclosure found non-classical elemental combinations within 179 unlabeled perovskites found to be synthesized in addition to existing ionic perovskites. These types include “covalent” perovskites containing two or more anions (e.g., CsIO₃, ClOLi₃) with higher covalent bonds in the bond, hydride perovskites containing hydrogen (e.g., CaCsH₃) and an anti-perovskite (e.g., SnNFe3) containing an anion on the B site instead of the C site in ABC₃ combination. The prediction of these three types of combinations is a new function of the perovskite synthesizability prediction method using a graph convolutional neural network and positive unlabeled learning model of the disclosure, which cannot be performed in a conventional model.

In FIG. 6, it was observed in c and d that a significant portion was out of scope. In addition, b in FIG. 6 shows that only the non-domain specific model predicted 101 stable crystals among the 179 found unlabeled crystals, thereby demonstrating the effects of domain-specific learning.

Although perovskites have been extensively studied, a in FIG. 6 shows that there remain many synthesizable element combinations that have yet to be discovered.

Compared to classical ionic perovskite (classical perovskite), anti-perovskite contains C, N, O, P at the B site and transition metal at the C site, and has a high CL score. Indeed, it was found that a significant number of virtual anti-perovskites have been synthesized before. This suggests that there may be more opportunities to discover anti-perovskites.

Anti-perovskites have shown many interesting properties, such as superconductivity and magnetism. The perovskite synthesizability prediction method using the graph convolutional neural network and the positive unlabeled learning model of the disclosure could suggest that 327 virtual anti-perovskites was able to be synthesized.

Also, synthesizable candidates for two technologically important applications were selected. Metal halide perovskites, namely CsPbI₃, RbPbI₃ and MAPbI₃(MA=CH₃NH₃⁺), have shown many promising applications in photovoltaics and light emitting diodes in the last decade.

However, these materials often contain toxic Pb. The semiconducting properties of these perovskites are mainly due to the p-orbitals of the diffusion atoms of the halides, so it is expected that there will be more semiconducting halide perovskites that can be accessed. The perovskite synthesizability prediction method using the graph convolutional neural network and the positive unlabeled learning model of the disclosure predicts that 98 virtual metal halides can be synthesized. A two-step density functional theory (DFT) procedure (HSE06 single point calculation after PBEsol relaxation) is used to further screen these materials by a band gap. As a result of the screening, it was found that 43 materials had band gaps. In particular, 12 candidates have band gaps between 0.7 and 2.0 eV, which can be promising for photovoltaics, including CL scores and energy above the hull. Most of the materials predicted here (8 out of 12 candidates) are thermodynamically stable (energy above the hull <0.1 eV/atom). In addition, the CL score values of all predictive materials overlap with the CL score distribution of positive data. Both materials (NPF₃ and RbCF₃) are very unstable (energy above the hull >1.0 eV/atom). The perovskite synthesizability prediction method using the graph convolutional neural networks and the positive unlabeled learning model of the disclosure has a relatively high true positive rate (TPR). It was confirmed that many of these constitutions contained non-standard chemical compounds (e.g., CsNaF₃ or RbOF₃) that could not be identified based on simple electron count considerations.

It was found that Li₃OCl, a lithium-rich anti-perovskite, had superionic conductivity for applications in solid battery electrolytes. Since the high conductivity was achieved due to the high Li concentration and the streamlined C-site diffusion pathway, it is expected that the conductivity can be transferred to other Li-rich anti-perovskites such as Li₃OBr. Although previously reported Li₃OBr and Li₃₀Cl are thermodynamically stable (0.012 eV/atom for Li₃OBr and 0.006 eV/atom for Li₃OCl), the newly predicted materials show low thermodynamic stability (>0.3 eV/atom). A similar discrepancy is also observed in the CL score distribution, indicating potential difficulties in thermodynamically synthesizing these materials, despite being further synthesizable based on the CL score. This suggests an intriguing possibility that the combined use of CL scores and thermodynamic matrices can compensate for the limitations of each approach and yield more reliable synthesizability predictions.

Perovskites represent a unique class of materials with desirable physical properties. To evaluate the synthesizability of perovskite materials, the perovskite synthesizability prediction method of the embodiments of the disclosure implemented domain-specific transfer PU learning. The perovskite synthesizability prediction method using the graph convolutional neural network and the positive unlabeled learning model of the disclosure showed the true positive rate of 0.957 in the sample, which is a significant improvement over conventional methods based on geometric factors (0.806 to 0.863). The literature was retrieved for 962 unlabeled crystals predicted to be synthesizable and it was found that 179 unlabeled crystals were synthesized, 943 crystal perovskites synthesized from three public crystal databases were added to a pool.

The same literature searched for the 1000 unlabeled crystals with the lowest synthesizability score yielded no synthetic cases, which further validated the perovskite synthesizability prediction method using the graph convolutional neural network and the positive unlabeled learning model of the disclosure.

Compared to a conventional empirical model based on ionic radius, which is most applicable to ionic perovskites, the perovskite synthesizability prediction method using the graph spiral neural network and the positive unlabeled learning model of the disclosure showed a general ability to evaluate the synthesizability in all prototypes of perovskites, including anti-perovskites, covalent perovskites, halides, and hydrides.

The perovskite synthesis possibility prediction method using the graph convolutional neural network and the positive unlabeled learning model of the disclosure can be useful for exploring targeted crystal spaces for different crystal families and application domains.

<Learning of a Graph Convolutional Neural Network Model>

The overall architecture of the graph convolutional neural network (convolutional neural network) is shown in c of FIG. 2. Vin and Ein are the input features of atomic and edge interaction for a model. The crystal graph structure is constructed by assigning edges to Voronoi neighbors within a radius of 7 Å of each atom. An atomic feature is constructed with a one-hot encoding method classified for each element, and an edge feature is constructed with Gaussian extension of the distance and the Voronoi solid angle, as shown in d of FIG. 2. These features are encoded with linear multiplication and softplus activation. Graph convolutional layers include neighbor edge and atom pooling to create new hidden features. Specifically, a new edge feature, Eout,i of edge i is generated by Equation (1).

E_{out,j}=σ(W·ϕ(V_{in,j},V_{in,k},E_{in,j}) (1)

Where σ is the softplus function, W is the linear multiplication weight, β is the bias, ϕ is the concatenation operator, and j, k are the two atoms connecting the edges.

A new atomic feature Vout,i for atom i is created by Equation (2).

$${V}_{out , i} = σ ( W \cdot ϕ ( {V}_{in , j} , {\sum }_{j}^{{n}_{neighbor}} \frac{{E}_{in , j}}{{n}_{neighbor}} ) + β ) \qquad ( 2 )$$

Where j is the index of the edge connected to atom i. Here the edge features are averaged and connected.

In c of FIG. 2, the box of “Dense, 64” with two input arrows represents the two convolution operators described above. 64 indicates that the output feature size is 64. “Dense, 64” with one input arrow indicates a simple activation layer for feature Fout.

Fₒᵤₜ=σ(W·Fᵢₙ+β) (3)

For boxes with “Linear,1”, linear multiplication is used in Equation (4) to produce a single element value.

Fₒᵤₜ=W·Fᵢₙ+β (4)

“Min Pool” represents a minimum pooling operation followed by a sigmoid operation. As discussed above, intermittent atomic and edge features are kept at an element size of 64. The model was trained with a batch size of 512 using the binary cross-entropy loss function with the Adam optimizer. The model was trained for 50 epochs, and the model with the lowest validation loss is selected.

<Hull Band Gap and Energy Calculation>

PAW-PBE pseudopotentials are used.

PAW potentials were selected as recommended by the MP database. Atomic positions and unit cell parameters are fully relaxed using a convergence criterion of 1.0×10⁻⁵ eV for energy and conjugate gradient descent of 0.05 eV/Å for force with a cutoff energy of 500 eV.

Brillouin zone is used as a k-point density of 1000 k-points per atom using python materials genomics (Pymatgen). To calculate the band gap using the relaxed structure, the HSE0669 hybrid density functional theory (DFT) function implemented in VASP68 with the mixing parameter of 0.2 was performed. For computational efficiency, a cut-off energy of 400 eV was used and a uniform reduction factor was used for the q-point grid of the exact exchange potential. Gamma-centered even K-points (k-point density of 1000k-points per an atom). For Brillouin zone integration, the tetrahedral method with BlÖchl correction was used. To calculate the energy above the hull, all relevant species of convex hull diagram were extracted from the material project (MP) database and PBEsol calculation was performed. The energy above the hull is obtained using computed energies and python materials genomics (Pymatgen).

Another embodiment of the disclosure may provide a recording medium on which the perovskite synthesizability prediction method is recorded as a code that is read and executed by a computer.

Although the technical idea of the disclosure described above has been specifically described in a preferred embodiment, it should be noted that the above embodiment is for explanation and not for limitation. In addition, those of ordinary skill in the technical field of the disclosure will be able to understand that various embodiments are possible within the scope of the technical spirit of the disclosure. Therefore, the true technical protection scope of the disclosure should be determined by the technical spirit of the appended claims.

What is claimed is:

1. A method for predicting perovskite synthesizability using graph convolutional neural networks and positive unlabeled learning, the method comprising:
performing pre-training for perovskite synthesizability prediction by inputting stored material data into a graph convolutional neural network model that calculates a perovskite synthesizability score;
performing retraining for the perovskite synthesizability prediction by inputting stored perovskite data to the graph convolutional neural network model; and
predicting the perovskite synthesizability by calculating the perovskite synthesizability score by randomly selecting unlabeled data from a perovskite data set as negative data and then applying the selected unlabeled data to the retrained graph convolutional neural network model.

2. The method of claim 1, wherein the graph convolutional neural network model is configured to receive an atomic feature and an edge feature of each of the material data and the perovskite data as an input value and calculate the synthesizability score of the material or the perovskite.

3. The method of claim 1, wherein the performing the pre-training for the perovskite synthesizability prediction comprises pre-training the graph convolutional neural network model by positive unlabeled learning that repeatedly calculates a synthesizability score by applying the graph convolutional neural network model after randomly selecting unlabeled material data included in the material data for which synthesizability is not determined to set the selected unlabeled material data to a negative indicating synthesis impossibility.

4. The method of claim 1, wherein the performing the retraining for the perovskite synthesizability prediction comprises retraining the graph convolutional neural network model by positive unlabeled learning that repeatedly calculates a synthesizability score by applying the pre-trained graph convolutional neural network model after randomly selecting unlabeled perovskite data included in the perovskite data for which synthesizability is not determined to set the selected unlabeled perovskite data to a negative indicating synthesis impossibility.

5. The method of claim 1, wherein the predicting perovskite synthesizability comprises predicting the perovskite synthesizability by performing positive unlabeled learning that repeatedly calculates synthesizability by randomly selecting unlabeled perovskite data from the perovskite data set as the negative data and then inputting the selected unlabeled perovskite data to the retrained graph convolutional neural network model, and averaging the synthesizability score for each perovskite data calculated in each data set by the positive unlabeled learning.

6. The method of claim 1, wherein the perovskite synthesizability is predicted as being synthesizable when the calculated perovskite synthesizability score is 0.5 or more.

7. A non-transitory recording medium in which a method for predicting perovskite synthesizability is recorded as a code that is read and executed by a computer, wherein the method for predicting perovskite synthesizability comprises:
performing pre-training for perovskite synthesizability prediction by inputting stored material data into a graph convolutional neural network model that calculates a perovskite synthesizability score;
performing retraining for the perovskite synthesizability prediction by inputting stored perovskite data to the graph convolutional neural network model; and
predicting the perovskite synthesizability by calculating the perovskite synthesizability score by randomly selecting unlabeled data from a perovskite data set as negative data and then applying the selected unlabeled data to the retrained graph convolutional neural network model.
